(19) United States
(12) Patent Application Publication
(10) Pub. No.: US 2020/0316153 A1
(43) Pub. Date: Oct. 8, 2020

(54) EXTERNALLY-USED TRADITIONAL CHINESE MEDICINE PREPARATION FOR TREATING RHINITIS, AND PREPARATION METHOD THEREFOR

(71) Applicant: LETANG MEDICAL INVESTMENT CO., LTD (DONG GUAN), Dongguan, Guangdong (CN)

(72) Inventor: Zhehao Liu, Dongguan, Guangdong (CN)

(21) Appl. No.: 16/310,507

(22) PCT Filed: Jun. 2, 2017
(86) PCT No.: PCT/CN2017/087029

§ 371 Date: Dec. 17, 2018

(30) Foreign Application Priority Data
Jun. 20, 2016 (CN) 201610440969.X

(51) Int. Cl. A61K 36/268; A61K 36/575; A61K 36/28; A61K 36/48; A61K 36/29; A61K 31/045; A61P 11/02

(52) U.S. Cl. CPC A61K 36/268; A61K 36/575; A61K 36/28; A61K 9/0043; A61K 36/29; A61K 31/045; A61P 11/02; A61K 36/48

(57) ABSTRACT

An externally-used traditional Chinese medicine preparation for treating rhinitis, and a preparation method therefor. The externally-used traditional Chinese medicine preparation is prepared by using the following components in parts by weight: 1 to 6 parts of herba asari, 8 to 12 parts of magnolia flowers, 6 to 20 parts of cocklebur fruits, 3 to 8 parts of small centipeda herb, 1 to 6 parts of cutch, 15 to 20 parts of roots of podophyllum hexandrum and 7 to 15 parts of borneol.

### TECHNICAL FIELD

The present invention relates to the technical field of traditional Chinese medicine preparations, in particular to a traditional Chinese medicine external preparation for treating rhinitis and a preparation method therefor.

### BACKGROUND OF THE INVENTION

When air temperature changes suddenly, acute rhinitis will occur easily as the nasal cavity fails to adapt to the change promptly. When a person has the bad living habits of pulling nose hair and digging the nose often, the skin of the nasal cavity will be torn and the hair follicle damaged, leading to rhinitis. In the case of improper medication, drugs such as nasal drops that contain a nasal mucosal vasoconstriction component will cause chronic poisoning of nasal mucosa. In a working or living environment which is dry or which is contaminated with dust or toxic substances, such as traffic fumes, decorative materials and food additives, the nasal mucosa will be irritated and damaged. And, the gradual annual rise in air temperature and the universal use of air conditioners are the main cause of allergic rhinitis.

Rhinitis, an inflammatory disease of the nasal cavity, is an inflammation of the nasal mucosa caused by viruses, bacteria, allergens, various physical and chemical factors, and certain systemic diseases. The main pathological changes of rhinitis are nasal mucosal congestion, swelling, exudation, hyperplasia, atrophy or necrosis, among others. Typical conditions of rhinitis usually manifest as nasal congestion, runny nose, sneezing, rhinitis-related headaches, dizziness, and the like. Rhinitis does not affect the nose alone. Long-term intermittent or alternating nasal congestion can cause dizziness and heavy feeling in the brain, affecting sleep, work and study. Moreover, the nasal discharge will often reflux to the pharynx, causing coughing and phlegm.

Western medicine holds that rhinitis is caused by infection with viruses and germs. The treatments usually used are antibiotics for sterilization and inflammation reduction, as well as surgery, and laser, etc. Although such treatments can take effect quickly, they are painful, palliative in nature, easily recurrent and expensive in medical costs.

Traditional Chinese medicine holds that the nose is the outside orifice of the lungs and the lungs open to the nose. If the qi of the lungs is abundant, the breath will be smooth. Rhinitis and sinusitis originate in the lungs, mostly due to weakness of the spleen and lungs, insufficiency of the qi of the lungs and inability to prevent exogenous pathogenic factors. Weakness of the spleen results in deficient transforming function of the spleen, retention of phlegm in the nose cavity, soaking of sinus mucosa, and nasosinusitis. The traditional Chinese medicine external preparations commonly used suffer from low therapeutic effect, long treatment period, and high recurrent rate, despite their weak toxic side effects.

Therefore, the prior art has yet to be improved and developed.

### SUMMARY OF THE INVENTION

In view of the above-mentioned drawbacks of the prior art, an object of the present invention is to provide a traditional Chinese medicine external preparation for treating rhinitis and a preparation method therefor, aiming to solve the problems of low therapeutic effect, long treatment period and high recurrence rate associated with existing traditional Chinese medicine external preparations.

The technical solutions of the present invention are as follows:

A traditional Chinese medicine external preparation for treating rhinitis, comprising the following components by weight: 1 to 6 parts of Herba Asari, 8 to 12 parts of Flos Magnoliae Liliflorae, 6 to 20 parts of siberial cocklebur fruit, 3 to 8 parts of herba centipedae, 1 to 6 parts of catechu, 15 to 20 parts of Radix Podophylli, and 7 to 15 parts of borneol.

The traditional Chinese medicine external preparation for treating rhinitis, comprising the following components by weight: 2 to 6 parts of Herba Asari, 9 to 11 parts of Flos Magnoliae Liliflorae, 10 to 15 parts of siberial cocklebur fruit, 6 to 8 parts of herba centipedae, 1 to 3 parts of catechu, 18 to 20 parts of Radix Podophylli, and 7 to 9 parts of borneol.

The traditional Chinese medicine external preparation for treating rhinitis, comprising the following components by weight: 2 parts of Herba Asari, 10 parts of Flos Magnoliae Liliflorae, 15 parts of siberial cocklebur fruit, 8 parts of herba centipedae, 2 parts of catechu, 20 parts of Radix Podophylli, and 8 parts of borneol.

The traditional Chinese medicine external preparation for treating rhinitis, comprising the following components by weight: 6 parts of Herba Asari, 8 parts of Flos Magnoliae Liliflorae, 16 parts of siberial cocklebur fruit, 5 parts of herba centipedae, 6 parts of catechu, 17 parts of Radix Podophylli, and 7 parts of borneol.

The traditional Chinese medicine external preparation for treating rhinitis, comprising the following components by weight: 4 parts of Herba Asari, 12 parts of Flos Magnoliae Liliflorae, 6 parts of siberial cocklebur fruit, 7 parts of herba centipedae, 5 parts of catechu, 18 parts of Radix Podophylli, and 10 parts of borneol.

A preparation method of any traditional Chinese medicine external preparation for treating rhinitis described above, comprising the steps of: according to the formula described above, selecting herba centipedae herbs, washing them to remove impurities, and pounding them to collect the juice; selecting the stems and the roots of Radix Podophylli, boiling them in yellow wine for 50 to 70 minutes, and then removing the dregs to collect the soup; selecting siberial cocklebur fruits and heating them in a hot-frying container with medium fire until the surface becomes dark yellow, the prickles become burnt and the interior becomes light yellow, taking the fruits out, grinding away the prickles, sieving the fruits clean and then pounding them to collect the juice; selecting the other four components of the preparation, i.e., Flos Magnoliae Liliflorae, catechu, Herba Asari, and borneol, respectively grounding them into powders, adding the powders to the medicinal liquor prepared above, stirring the resulting mixture evenly, and filtering off the dregs with cotton filter, thus obtaining the traditional Chinese medicine external preparation.

The preparation method for the traditional Chinese medicine external preparation for treating rhinitis, comprising boiling the stems and the roots of Radix Podophylli in yellow wine for 60 minutes, and then removing the dregs to collect the soup.

Beneficial effects: The traditional Chinese medicine external preparation for treating rhinitis of the present invention uses purely natural green medicinal materials, is convenient to use, can directly reach the focus, can be rapidly absorbed, has strong effect without hormone-type toxic and side effects, and is safe and reliable. It has remarkable therapeutic effects on various types of rhinitis, with a short treatment period and a low recurrence rate.

### DETAILED DESCRIPTION OF THE INVENTION

The present invention provides a traditional Chinese medicine external preparation for treating rhinitis and a preparation method therefor. In order to make the object, the technical solutions and the effects of the present invention more clear and concrete, the present invention will be further described in detail below. It is appreciated that the specific embodiments described herein are merely illustrative of the present invention and are not intended to limit the present invention.

A preferred embodiment of the traditional Chinese medicine external preparation for treating rhinitis of the present invention comprises the following components by weight: 1 to 6 parts of Herba Asari, 8 to 12 parts of Flos Magnoliae Liliflorae, 6 to 20 parts of siberial cocklebur fruit, 3 to 8 parts of herba centipedae, 1 to 6 parts of catechu, 15 to 20 parts of Radix Podophylli, and 7 to 15 parts of borneol. The traditional Chinese medicine external preparation for treating rhinitis of the present invention uses purely natural green medicinal materials, is convenient to use, can directly reach the focus, can be rapidly absorbed, has strong effect without hormone-type toxic and side effects, and is safe and reliable. It has remarkable therapeutic effects on various types of rhinitis, with a short treatment period and a low recurrence rate.

Another preferred embodiment of the traditional Chinese medicine external preparation for treating rhinitis of the present invention comprises the following components by weight: 2 to 6 parts of Herba Asari, 9 to 11 parts of Flos Magnoliae Liliflorae, 10 to 15 parts of siberial cocklebur fruit, 6 to 8 parts of herba centipedae, 1 to 3 parts of catechu, 18 to 20 parts of Radix Podophylli, and 7 to 9 parts of borneol. Using the above preferred formula, the traditional Chinese medicine external preparation prepared has stronger therapeutic effect, shorter treatment period and lower recurrent rate.

Yet another preferred embodiment of the traditional Chinese medicine external preparation for treating rhinitis of the present invention comprises the following components by weight: 2 parts of Herba Asari, 10 parts of Flos Magnoliae Liliflorae, 15 parts of siberial cocklebur fruit, 8 parts of herba centipedae, 2 parts of catechu, 20 parts of Radix Podophylli, and 8 parts of borneol. Using the above preferred formula, the traditional Chinese medicine external preparation prepared has further stronger therapeutic effect, and shorter treatment period and lower recurrent rate.

Still another preferred embodiment of the traditional Chinese medicine external preparation for treating rhinitis of the present invention comprises the following components by weight: 6 parts of Herba Asari, 8 parts of Flos Magnoliae Liliflorae, 16 parts of siberial cocklebur fruit, 5 parts of herba centipedae, 6 parts of catechu, 17 parts of Radix Podophylli, and 7 parts of borneol. Using the above preferred formula, the traditional Chinese medicine external preparation prepared also has the excellent properties of strong therapeutic effect, short treatment period and low recurrent rate.

Still another preferred embodiment of the traditional Chinese medicine external preparation for treating rhinitis of the present invention comprises the following components by weight: 4 parts of Herba Asari, 12 parts of Flos Magnoliae Liliflorae, 6 parts of siberial cocklebur fruit, 7 parts of herba centipedae, 5 parts of catechu, 18 parts of Radix Podophylli, and 10 parts of borneol. Using the above preferred formula, the traditional Chinese medicine external preparation prepared also has the excellent properties of strong therapeutic effect, short treatment period and low recurrent rate.

The Herba Asari used in the present invention is prepared from the dried roots and rhizomes of Herba Asari Heterotropoidedis, Herba Asari sieboldii or Herba Asari Sieboldii of the Aristolochiaceae family. The herba asari has the effect of relieving exterior syndrome and dissipating coldness, expelling wind and stopping pain, inducing resuscitation, and warming the lungs and dissolving the phlegm. The herba asari mainly treats anemofrigid cold, headache, toothache, rheumatism, nasosinusitis, and cough due to pulmonary cold.

The Flos Magnoliae Liliflorae used in the present invention is prepared from Magnolia liliflora, a deciduous tree plant. The flower bud of the plant is warm in nature and pungent in taste, and acts on the lung and stomach meridians. It has a dissipating and warming effect. It's fragrance is migratory up to the head and the face. It is good at relieving a stuffy nose and is a good drug for nasosinusitis and headache. When appropriately dosed, it can be used in both cold and hot conditions. It has a very high medicinal value and a very good therapeutic effect in both cases of internal and external use. The siberial cocklebur fruit used in the present invention is the involucrum fruit of the yearly herbal plant Xanthium sibiricum belonging to Xanthium genus, Compositae family. Siberial cocklebur fruit is a Chinese herbal medicine and has the effect of dispersing wind, dispelling dampness, and inducing resuscitation.

The herba centipedae used in the present invention belongs to Hydrocotyle genus, Compositae family. It is pungent in taste and warm in nature, and acts on the lung meridian. It has the effects of dispelling wind and cold, relieving a stuffy nose, stopping cough, eliminating toxicity and stopping itching, among others. It is pungent, acrid and invigorating, can open up the lung meridian up to the brain. Therefore, such conditions as asthma due to excessive phlegm, qi blockage and obstruction, stuffy nose and rhinopolypus, distension, nebula in the eyes, cold-evil in the head, and head wind and brain pain, will all be treated thanks to the pungent, warm and invigorating effects. The catechu used in the present invention is the dried decoction prepared from the peeled branches and stems of Acacia catechu plant of Leguminosae family. In winter, the branches and stems are harvested, peeled, and chopped into chunks. The chunks are decocted with water, and the decoction is concentrated and dried. The Acacia catechu plant of Leguminosae family is distributed in southern part of Yunnan province and is also planted in Hainan province. The catechu decoction has the efficacy of promoting blood circulation and relieving pain, stopping bleeding and growing muscle, restraining dampness and sore, clearing the lungs and removing phlegm. It is used to treat fall injury and pain, traumatic bleeding, blood vomiting and nose bleeding, non-cicatrizing ulcers, eczema, wet sores, and cough with lung heat.

The Radix Podophylli used in the present invention is one of the “Taibai Seven-series Herbs” and has a miraculous anti-cancer effect. Its rhizome, fibrous root and fruit can all be used as medicine. The rhizome has the effect of removing rheumatism, benefiting qi and blood, relaxing tendon, and stopping cough; the fruit has the effects of promoting the secretion of saliva and benefiting the stomach, invigorating the spleen and regulating the flow of qi, stopping cough and removing phlegm, and is effective against numbness and irregular menstruation. The root and the rhizome can promote the flow of qi and the circulation of blood, stopping pain and cough, dispelling wind and removing dampness. The root and the rhizome can be used for treating rheumatism, cough and asthma, traumatic injuries and irregular menstruation. The fruit is sweet and mild in nature, can promote blood circulation and restore menstrual flow, stop cough and relieve asthma, invigorate the spleen and regulate the flow of qi. The fruit can be used for treating internal lesion caused by overexertion, cough with asthma, backache, irregular menstruation, mazischesis, leukorrheal diseases, and cervical cancer.

The borneol used in the present invention is pungent and bitter in taste, and slightly cold in nature; and it acts on the heart, liver and lung meridians. It is mildly fragrant, soothing and relaxing, and has the efficacy of inducing resuscitation, clearing away heat and dispersing toxicity, clearing the eyes and removing nebula in the eyes. It is mainly used for treating fever, high fever, fainting, stroke, coma due to blocking of the respiratory system, convulsive epilepsy, blocking of the upper orifices by summer-heat and damp, haryngeal tumefaction, deafness, mouth sores, tooth swelling, sores and ulcers, hot and swelling eyes, and conjunctivitis.

Traditional Chinese medicine is effective in treating rhinitis with less toxic and side effects, and the treatment is radical. Traditional Chinese medicine external preparation can avoid the potential toxicity of internal preparation. The medicine directly acts on the nasal cavity, avoiding the trauma caused by laser, surgery and plasma to the nasal cavity. It is only necessary to apply the medicinal liquor in the nasal cavity, without the need to take medicine or take injection, and the effect is obvious.

On the basis of the above traditional Chinese medicine external preparation, the present invention further provides a method for preparing any of the traditional Chinese medicine external preparation for treating rhinitis described above, comprising the steps of: according to the formula described above, selecting fresh herba centipedae herbs, washing them to remove impurities, and pounding them to collect the juice; selecting the stems and the roots of Radix Podophylli, boiling them in a suitable amount of yellow wine for 50 to 70 minutes (for example, 60 minutes), and then removing the dregs to collect the soup; selecting siberial cocklebur fruits and heating them in a hot-frying container with medium fire until the surface becomes dark yellow, the prickles become burnt and the interior becomes light yellow, taking the fruits out, grinding away the prickles, sieving the fruits clean and then pounding them to collect the juice; selecting the other four components of the preparation, i.e., Flos Magnoliae Liliflorae, catechu, Herba Asari, and borneol, respectively grounding them into powders, adding the powders to the medicinal liquor prepared above, stirring the resulting mixture evenly, and filtering off the dregs with cotton filter, thus obtaining the traditional Chinese medicine external preparation.

The following is a description of the method for using the traditional Chinese medicine external preparation of the present invention: firstly, the nasal cavity is cleaned with pure water or physiological saline, then the moisture in the nasal cavity with a cotton ball, and finally smearing the traditional Chinese medicine external preparation into the nasal cavity with a push hose to allow the preparation to be absorbed.

Usage and Dosage: An appropriate amount of the above-mentioned traditional Chinese medicine external preparation is evenly applied to the mucosa of the nasal cavity, then one hour later the residual medicine left after nasal cavity absorption is washed clean. The preparation is used once every other day in one week as one course of treatment. The symptoms can be obviously relieved after using the preparation twice, and various types of acute and chronic rhinitis can be cured in two courses of treatment. In the present invention, the raw traditional Chinese medicinal materials used are those which have been pretreated by regular pharmacies or hospitals, and they can be directly used to prepare the traditional Chinese medicine external preparation in the present invention. The pretreatment of raw traditional Chinese medicinal materials is conducted, according to the particular properties of the raw medicinal materials or the medicinal material crude slices, by subjecting the materials to suitable processing steps of washing, soaking, cutting, selecting, frying, drying, among others, thus obtaining the intermediate or semi-finished products of the traditional Chinese medicinal materials that conform to certain quality specifications.

Commonly, botanical and animal medicinal materials are mingled with various impurities, and mineral medicinal materials are mingled with mud and sand. Therefore, various impurities are removed by picking, screening, air selecting, washing, bleaching, among other methods, and the non-medicinal parts are removed by removing stems, roots, branches, skins, husks, furs, kernels, among other methods.

The traditional Chinese medicine external preparation for treating rhinitis according to the present invention is formulated according to the formulating principle of “Junchen Zuozhi (literally, Monarch, Minister, Assistant and Guide)”. According to the ancient book of Neijing, The medicine mainly responsible for a disease is the Monarch, the medicine supporting the Monarch is the Minister, and the medicine assisting the Minister is the Assistant. Based on this formulating principle, the preparation of the present invention is formulated by using qi, blood, yin and yang as the basis of basic prescription. And, the preparation of the present invention is a compound prescription of traditional Chinese medicine formulated from multiple aspects to cater to the intricacies of diseases and the various characteristics of the human body itself.

Compared with the prior art, the present invention has the following advantages: the traditional Chinese medicine external preparation for treating rhinitis of the present invention uses purely natural green medicinal materials which are low in price; it can be used conveniently, has no hormone-type toxic and side effects, can enhance the immunity of the body, and can reduce recurrence; it is safe and reliable, and is remarkably effective, with an excellent therapeutic effect on rhinitis. The traditional Chinese medicine external preparation does not require traumatic treatment by laser, surgery, or plasma, and only needs to be applied into the nasal cavity, without the need to take medicine or take injection. In combination with traditional Chinese proved recipes, it can restore the normal functioning of the nasal cavity in a short time.

Clinical Trials and Test Results

I. 150 patients with allergic rhinitis, sinusitis, nasal polyps, turbinate hypertrophy, olfactory disorder, etc. were selected for clinical observation and treatment, including 89 males and 61 females, the oldest being 75 years old and the youngest being 3 years old.

2. All the cases manifested the main clinical symptoms as follows: clear phlegm, nasal congestion, nasal itching, intermittent sneezing, accompanied with headache, dizziness, and itchy eyes.

III. Criteria for assessing the efficacy:

(1) cured: all of the above-mentioned symptoms of rhinitis disappeared, and all of the auxiliary examination indicators were normal;

(2) improved: most of the above-mentioned symptoms of rhinitis disappeared, and most of the auxiliary examination indicators were normal;

(3) effective: some of the above-mentioned symptoms of rhinitis disappeared, and some of the auxiliary examination indicators were normal;

(4) ineffective: The above-mentioned symptoms of rhinitis and the auxiliary examination indicators were not improved, or even were aggravated.

IV. Treatment method: The traditional Chinese medicine external preparation for treating rhinitis were applied into the nasal cavity after supper every other day. The nasal cavity was washed 1 hour after application. One course of treatment lasted 1 week, and the treatment lasted 2 courses.

V. Treatment results: 123 cases were clinically cured, accounting for 82%; 16 cases improved, accounting for 10.7%; 8 cases effective, accounting for 5.3%; 3 cases ineffective, accounting for 2%. The total effective rate was 98%. It can be seen from the above clinical test results that the total effective rate of the traditional Chinese medicine external preparation for treating rhinitis of the present invention can be up to 98%. The preparation has remarkable curative effect and high safety without side effects, and is an ideal and effective medicine for treating rhinitis.

In summary of the above, the present invention discloses the traditional Chinese medicine external preparation for treating rhinitis and the preparation method thereof. The traditional Chinese medicine external preparation comprises the following components by weight: 1 to 6 parts of Herba Asari, 8 to 12 parts of Flos Magnoliae Liliflorae, 6 to 20 parts of siberial cocklebur fruit, 3 to 8 parts of herba centipedae, 1 to 6 parts of catechu, 15 to 20 parts of Radix Podophylli, and 7 to 15 parts of borneol. The traditional Chinese medicine external preparation for treating rhinitis of the present invention uses purely natural green medicinal materials, is convenient to use, can directly reach the focus, can be rapidly absorbed, has strong effect without hormone-type toxic and side effects, and is safe and reliable. It has remarkable therapeutic effects on various types of rhinitis, with a short treatment period and a low recurrence rate.

It is appreciated that the use of the present invention is not limited to the above-described examples, and those skilled in the art can make modifications or changes in accordance with the above description, all of which being within the scope of protection of the appended claims.

1. A traditional Chinese medicine external preparation for treating rhinitis, comprising the following components by weight: 1 to 6 parts of Herba Asari, 8 to 12 parts of Flos Magnoliae Liliflorae, 6 to 20 parts of siberial cocklebur fruit, 3 to 8 parts of herba centipedae, 1 to 6 parts of catechu, 15 to 20 parts of Radix Podophylli, and 7 to 15 parts of borneol.

2. The traditional Chinese medicine external preparation for treating rhinitis according to claim 1, comprising the following components by weight: 2 to 6 parts of Herba Asari, 9 to 11 parts of Flos Magnoliae Liliflorae, 10 to 15 parts of siberial cocklebur fruit, 6 to 8 parts of herba centipedae, 1 to 3 parts of catechu, 18 to 20 parts of Radix Podophylli, and 7 to 9 parts of borneol.

3. The traditional Chinese medicine external preparation for treating rhinitis according to claim 1, comprising the following components by weight: 2 parts of Herba Asari, 10 parts of Flos Magnoliae Liliflorae, 15 parts of siberial cocklebur fruit, 8 parts of herba centipedae, 2 parts of catechu, 20 parts of Radix Podophylli, and 8 parts of borneol.

4. The traditional Chinese medicine external preparation for treating rhinitis according to claim 1, comprising the following components by weight: 6 parts of Herba Asari, 8 parts of Flos Magnoliae Liliflorae, 16 parts of siberial cocklebur fruit, 5 parts of herba centipedae, 6 parts of catechu, 17 parts of Radix Podophylli, and 7 parts of borneol.

5. The traditional Chinese medicine external preparation for treating rhinitis according to claim 1, comprising the following components by weight: 4 parts of Herba Asari, 12 parts of Flos Magnoliae Liliflorae, 6 parts of siberial cocklebur fruit, 7 parts of herba centipedae, 5 parts of catechu, 18 parts of Radix Podophylli, and 10 parts of borneol.

6. A preparation method for the traditional Chinese medicine external preparation for treating rhinitis according to claim 1, comprising the steps of: according to the formula described, selecting herba centipedae herbs, washing them to remove impurities, and pounding them to collect the juice; selecting the stems and the roots of Radix Podophylli, boiling them in yellow wine for 50 to 70 minutes, and then removing the dregs to collect the soup; selecting siberial cocklebur fruits and heating them in a hot-frying container with medium fire until the surface becomes dark yellow, the prickles become burnt and the interior becomes light yellow, taking the fruits out, grinding away the prickles, sieving the fruits clean and then pounding them to collect the juice; selecting the other four components of the preparation, i.e., Flos Magnoliae Liliflorae, catechu, Herba Asari, and borneol, respectively grounding them into powders, adding the powders to the medicinal liquor prepared above, stirring the resulting mixture evenly, and filtering off the dregs with cotton filter, thus obtaining the traditional Chinese medicine external preparation.

7. The preparation method for the traditional Chinese medicine external preparation for treating rhinitis according to claim 6, comprising boiling the stems and the roots of Radix Podophylli in yellow wine for 60 minutes, and then removing the dregs to collect the soup.
